SEQUENCE LISTING (24 sequences)

SEQ ID NO: 1 - DNA, 25 bp, synthetic construct
  source 1..25: mol_type = other DNA
agcaatagcatcacaaatttcacaa

SEQ ID NO: 2 - DNA, 29 bp, synthetic construct
  source 1..29: mol_type = other DNA
ccagacatgataagatacattgatgagtt

SEQ ID NO: 3 - DNA, 36 bp, synthetic construct
  source 1..36: mol_type = other DNA
agcatttttttcactgcattctagttgtggtttgtc

SEQ ID NO: 4 - DNA, 15 bp, synthetic construct
  source 1..15: mol_type = other DNA
gaggcgggcggatca

SEQ ID NO: 5 - DNA, 27 bp, synthetic construct
  source 1..27: mol_type = other DNA
cccggctaatttttgtatttttagtag

SEQ ID NO: 6 - DNA, 24 bp, synthetic construct
  source 1..24: mol_type = other DNA
cagcctggccaacatggtgaaacc

SEQ ID NO: 7 - DNA, 18 bp, synthetic construct
  source 1..18: mol_type = other DNA
ttagaacggcccgcagtc

SEQ ID NO: 8 - DNA, 21 bp, synthetic construct
  source 1..21: mol_type = other DNA
ccttgagacccgagagttcac

SEQ ID NO: 9 - DNA, 75 bp, synthetic construct
  source 1..75: mol_type = other DNA
ggaggtacagcttggatttcccaagagtcctttgatgtagatgaaacagacagcggggcagggttgaagtggaag

SEQ ID NO: 10 - protein, 6 aa, synthetic construct
DVDETD

SEQ ID NO: 11 - DNA, 73 bp, synthetic construct
  source 1..73: mol_type = other DNA
ggaggtacagcttggatttcccaagagtcctttgatgtagatgaaacagagagcggggcaggttgaagtggag

SEQ ID NO: 12 - protein, 6 aa, synthetic construct
DVDETE

SEQ ID NO: 13 - DNA, 21 bp, synthetic construct
  source 1..21: mol_type = other DNA
aatggacctgttgacctgaaa

SEQ ID NO: 14 - DNA, 18 bp, synthetic construct
  source 1..18: mol_type = other DNA
cacggcaggcctgaataa

SEQ ID NO: 15 - DNA, 20 bp, synthetic construct
  source 1..20: mol_type = other DNA
gactggtggcctggaaatag

SEQ ID NO: 16 - DNA, 20 bp, synthetic construct
  source 1..20: mol_type = other DNA
cgcctccttcccaactttat

SEQ ID NO: 17 - DNA, 21 bp, synthetic construct
  source 1..21: mol_type = other DNA
gtctgtgcccaaatcaacaag

SEQ ID NO: 18 - DNA, 22 bp, synthetic construct
  source 1..22: mol_type = other DNA
gagtccgagattgtcattaccc

SEQ ID NO: 19 - DNA, 22 bp, synthetic construct
  source 1..22: mol_type = other DNA
cctggaacctggatcacataat

SEQ ID NO: 20 - DNA, 20 bp, synthetic construct
  source 1..20: mol_type = other DNA
tactcccagtccacttctct

SEQ ID NO: 21 - DNA, 20 bp, synthetic construct
  source 1..20: mol_type = other DNA
aaggtgaaggtcggagtcaa

SEQ ID NO: 22 - DNA, 20 bp, synthetic construct
  source 1..20: mol_type = other DNA
aatgaaggggtcattgatgg

SEQ ID NO: 23 - DNA, 20 bp, synthetic construct
  source 1..20: mol_type = other DNA
gactgtggttgcttcatgct

SEQ ID NO: 24 - DNA, 20 bp, synthetic construct
  source 1..20: mol_type = other DNA
acgtgaatggtcttcagcag

(19) United States
(12) Patent Application Publication
(10) Pub. No.: US 2025/0163459 A1
(43) Pub. Date: May 22, 2025

(54) IMPROVED CELL LINES AND METHODS FOR THE PRODUCTION OF ADENO-ASSOCIATED VECTORS

(71) Applicant: CEVEC PHARMACEUTICALS GMBH, Köln (DE)

(72) Inventors: Michelle Hussong, Köln (DE); Tobias Bonifert, Köln (DE); Helmut Kewes, Köln (DE)

(21) Appl. No.: 18/841,934

(22) PCT Filed: Mar. 1, 2023
(86) PCT No.: PCT/EP2023/055118

§ 371 Date: Aug. 27, 2024

(30) Foreign Application Priority Data
Mar. 2, 2022 (EP) 22159636.4
Nov. 9, 2022 (EP) 22206412.3

(51) Int. Cl. C12N 15/86; C12N 5/071; C12N 9/22; C12N 9/64

(52) U.S. Cl. CPC C12N 15/86; C12N 5/0686; C12N 9/22; C12N 9/6472; C12Y 304/22056; C12N 2510/02; C12N 2750/14122; C12N 2750/14143; C12N 2750/14152

(57) ABSTRACT

The present invention relates to cell lines in which DNA fragmentation is inhibited, uses of cell lines in which DNA fragmentation is inhibited for the production of Adeno-associated virus (AAV), related methods of producing AAV, and methods of producing AAV, comprising the step of exposing the cells in which AAV is produced to an inhibitor of DNA fragmentation during the AAV production phase.

The present invention relates to cell lines in which DNA fragmentation is inhibited, uses of cell lines in which DNA fragmentation is inhibited for the production of Adeno-associated virus (AAV), related methods of producing AAV, and methods of producing AAV, comprising the step of exposing the cells in which AAV is produced to an inhibitor of DNA fragmentation during the AAV production phase.

In recent years, a rapid increase in the number of gene therapy trials and products based on Adeno-associated virus (AAV)-derived vectors could be observed. Advantages of AAV vectors in gene therapy include an advantageous safety profile, the fact that AAV is not pathogenic, i.e., is not associated with any disease, the stable expression of transgenes, and the possibility of transducing dividing as well as non-dividing cells. However, exhaustive quality control (QC) must be performed on all AAV-derived products to ensure their efficiency and safety.

A major challenge for the production of AAV-based preparations is the identification, characterization, and control of process- and product-related impurities that may occur in the already purified products. Impurities that remain after vector purification include residual amounts of chemical components, proteins and nucleic acids derived from the cell culture system in which the vector product was generated. Nucleic acid impurities include residual DNA, specifically host cell DNA and/or plasmid DNA, wherein residual host cell DNA may exist in two forms: (1) as nuclease-sensitive process-related contamination, i.e., non-specifically co-purified with the desired AAV vector product; and (2) as nuclease-resistant product-related contamination, i.e., encapsidated in AAV particles. This latter type of product impurity cannot be removed by conventional “downstream process” methods.

Residual DNA impurities pose a significant safety hazard because the DNA might encode proteins or regulatory RNAs and even trigger immune toxicity themselves via TLR9 activation. Thus, e.g., the Food and Drug Administration (FDA) recommendations are that the level of residual cell-substrate DNA should be below 10 ng per dose and a median DNA size of 200 bp or lower.

The encapsidation of fragments of mammalian producer cell genomic DNA has been reported to be generated at a frequency of 1% to 3% of AAV genome-containing particles. However, the mechanism of the packaging of fragments of host cell DNA within AAV particles is not yet understood.

Based on close similarity with the desired vector product, it is difficult to eliminate AAV packaged host cell DNA impurities by vector purification methods. Gradient centrifugation can remove AAV packaged nucleic acid impurities that differ significantly in length from the vector genome. However, there is so far no general technical solution to avoid, reduce and/or remove nuclease-resistant product-related nucleic acid impurities.

Accordingly, the technical problem underlying the present invention is the provision of means for the avoidance, reduction and/or removal of nuclease-resistant product-related nucleic acid impurities.

The solution to the above technical problem is achieved by the embodiments characterized in the claims.

In particular, in a first aspect, the present invention relates to a cell line in which DNA fragmentation is inhibited.

In preferred embodiments, the cell line in which DNA fragmentation is inhibited is used to produce Adeno-associated virus (AAV). More preferably, the cell line in which DNA fragmentation is inhibited is an AAV producer cell line.

In a second aspect, the present invention relates to the use of a cell line in which DNA fragmentation is inhibited for the production of Adeno-associated virus (AAV).

In preferred embodiments, the cell line used for the production of AAV is an AAV producer cell line.

In preferred embodiments, DNA fragmentation is (i) DNA fragmentation caused by programmed cell death, or (ii) DNA fragmentation caused by DNA strand breaks that are induced by Caspase 3/DFF40.

In preferred embodiments, DNA fragmentation that is inhibited in the present invention is low molecular weight DNA fragmentation, e.g. DNA fragmentation resulting in DNA fragments of at most 20 kB (kilobases) length.

The production of recombinant AAV inter alia requires the expression of AAV Rep and Cap proteins, usually encoded by the AAV genome, for production of recombinant virus supplied in trans. Further, helper genes are used which can be derived from different helper viruses, the most common being helper virus genes taken from Adenovirus (AV), such as E1A, E1B, E2A, E4orf6, or VA RNA, or helper virus genes taken from herpes simplex virus. Furthermore, a transfer vector containing the gene of interest (GOI) flanked by AAV inverted terminal repeat sequences (ITRs) is needed.

The most common system for the production of recombinant AAV relies on the introduction of all genes necessary for AAV production into the production cells by transient transfection. Transient transfection usually requires a two- or three-plasmid system: transfer vector containing gene of interest; pHelper with adenoviral helper functions; and pAAV-Rep2CapX (CapX=capsid function of different AAV serotypes) supplying the capsid and replicase functions.

Alternatively, some or all of the genes necessary for AAV production can be stably integrated into the host cell genome.

Accordingly, the term “AAV producer cell line” as used herein relates to any cell line in which some or all of the genes encoding the components necessary for the production of AAV are either transiently expressed in said cell line or are stably integrated into the cell genome. Further, the term “cell line used to produce AAV” as used herein relates to any cell line capable of producing a detectable amount of AAV.

Preferably, the genes encoding the components necessary for the production of AAV are selected from the group consisting of genes encoding the AAV Cap proteins VP1, VP2, and VP3; genes encoding the AAV Rep proteins Rep78, Rep68, Rep52, and Rep40; genes encoding the adenoviral helper functions E4orf6, E2A and VA-RNA; genes encoding the Ad5 helper genes E1A and E1B; and the gene of interest (GOI) flanked by AAV inverted terminal repeat sequences (ITRs). More preferably, the genes encoding the components necessary for the production of AAV include the genes encoding the AAV Cap proteins VP1, VP2, and VP3; a gene encoding the AAV Rep protein Rep78 or Rep68, a gene encoding the AAV Rep protein Rep52 or Rep40; a gene encoding the adenoviral helper function E4orf6, and the gene of interest (GOI) flanked by AAV ITRs. In specific embodiments, the genes encoding the components necessary for the production of AAV additionally include at least 1, at least 2, at least 3, at least 4, at least 5, or all of the genes encoding the AAV Rep protein Rep78, Rep68, Rep52 and Rep40; and the genes encoding the adenoviral helper functions E2A, VA-RNA, and DBP (DNA binding protein); and the genes encoding the Ad5 helper genes E1A and E1B.

Thus, in specific embodiments, in the AAV producer cell lines according to the present invention, at least 1, at least 2, at least 3, at least 4, at least 5, at least 6, at least 7, at least 8, at least 9, at least 10, at least 11, or all of the following genes are transiently expressed in said cell lines or are stably integrated into the cell genome:
the genes encoding the AAV Cap proteins VP1, VP2, and VP3;
a gene or genes encoding either one or both of the AAV Rep proteins Rep78 or Rep68;
a gene or genes encoding either one or both of the AAV Rep proteins Rep52 or Rep40;
the gene of interest (GOI) flanked by AAV ITRs; and
optionally, a gene or genes encoding either one, either two, either three, or all of the adenoviral helper functions E1A, E1B, DBP, E4orf6 and E2A.

In related specific embodiments, in the AAV producer cell lines according to the present invention, the following genes are transiently expressed in said cell lines or are stably integrated into the host cell genome:
(i) the genes encoding the AAV Cap proteins VP1, VP2, and VP3; a gene encoding the AAV Rep protein Rep78 or Rep68, a gene encoding the AAV Rep protein Rep52 or Rep40; and the gene of interest (GOI) flanked by AAV ITRs; or
(ii) the genes encoding the AAV Cap proteins VP1, VP2, and VP3; a gene encoding the AAV Rep protein Rep78 or Rep68, a gene encoding the AAV Rep protein Rep52 or Rep40; a gene encoding the adenoviral helper function E4orf6, and the gene of interest (GOI) flanked by AAV ITRs; or
(iii) the genes encoding the AAV Cap proteins VP1, VP2, and VP3; a gene encoding the AAV Rep protein Rep78 or Rep68, a gene encoding the AAV Rep protein Rep52 or Rep40; genes encoding the adenoviral helper functions E4orf6 and E2A, and the gene of interest (GOI) flanked by AAV ITRs; or
(iv) the genes encoding the AAV Cap proteins VP1, VP2, and VP3; a gene encoding the AAV Rep protein Rep78 or Rep68, a gene encoding the AAV Rep protein Rep52 or Rep40; genes encoding the adenoviral helper functions E1A, E1B, E4orf6 and E2A, and the gene of interest (GOI) flanked by AAV ITRs; or
(v) the genes encoding the AAV Cap proteins VP1, VP2, and VP3; the genes encoding the AAV Rep proteins Rep78, Rep68, Rep52 and Rep40; the genes encoding the adenoviral helper functions E4orf6 and E2A; and the gene of interest (GOI) flanked by AAV ITRs;
(vi) the genes encoding the AAV Cap proteins VP1, VP2, and VP3; the genes encoding the AAV Rep proteins Rep78, Rep68, Rep52 and Rep40; the genes encoding the adenoviral helper functions E1A, E1B, E4orf6 and E2A; and the gene of interest (GOI) flanked by AAV ITRs;
(vii) the genes encoding the AAV Cap proteins VP1, VP2, and VP3; a gene encoding the AAV Rep protein Rep78 or Rep68, a gene encoding the AAV Rep protein Rep52 or Rep40; genes encoding the adenoviral helper functions E4orf6 and DBP, and the gene of interest (GOI) flanked by AAV ITRs; or
(viii) the genes encoding the AAV Cap proteins VP1, VP2, and VP3; a gene encoding the AAV Rep protein Rep78 or Rep68, a gene encoding the AAV Rep protein Rep52 or Rep40; genes encoding the adenoviral helper functions E1A, E1B, E4orf6 and DBP, and the gene of interest (GOI) flanked by AAV ITRs; or
(ix) the genes encoding the AAV Cap proteins VP1, VP2, and VP3; the genes encoding the AAV Rep proteins Rep78, Rep68, Rep52 and Rep40; the genes encoding the adenoviral helper functions E4orf6 and DBP; and the gene of interest (GOI) flanked by AAV ITRs; or
(x) the genes encoding the AAV Cap proteins VP1, VP2, and VP3; the genes encoding the AAV Rep proteins Rep78, Rep68, Rep52 and Rep40; the genes encoding the adenoviral helper functions E1A, E1B, E4orf6 and DBP; and the gene of interest (GOI) flanked by AAV ITRs.

AAV in the present invention is not limited to particular AAV serotypes. Thus, AAV can be selected from any AAV serotype, e.g. from the group consisting of AAV serotype 1 (AAV1), AAV2, AAV3, AAV4, AAV5, AAV6, AAV7, AAV8, AAV9, AAV10, AAV11, AAVDJ, AAVDJ8, AAVrh10, hybrids of two or more different of said serotypes, and said serotypes having mutations that alter the tropism of the AAV serotype. Preferably, AAV is selected from the group consisting of AAV2, AAV5, AAV6, AAV8, and AAV9, wherein AAV8 is particularly preferred.

The cell lines according to the present invention can be any suitable cell line known in the art. However, in preferred embodiments, said cell line is selected from the group consisting of CAP cells, AGE1.hn, HEK293, PER.C6, NSO1, COS-7, BHK, CHO, CV1, VERO, HeLa, MDCK, BRL3A, W138, and HepG2 cells, wherein CAP cells or HEK293 cells are particularly preferred.

According to the present invention as defined in the above first and second aspects, encapsidation of host cell DNA is inhibited by inhibiting DNA fragmentation, preferably low molecular weight DNA fragmentation, which can for example be caused (i) by programmed cell death, or (ii) by DNA strand breaks that are induced by Caspase 3/DFF40, in the cells used for AAV production.

As used herein, the term “programmed cell death” includes apoptosis, parthanatos, and pyroptosis. Accordingly, the term “DNA fragmentation caused by programmed cell death” as used herein includes apoptotic DNA fragmentation, parthanatotic DNA fragmentation, and pyroptotic DNA fragmentation. In this context, apoptotic DNA fragmentation is of particular relevance in the present invention.

Preferably, inhibition of DNA fragmentation encompasses any direct or indirect inhibition of endonucleases. For example, direct inhibition of endonucleases can be achieved by genetic modification, chemical inhibition, protein depletion of (among others) endonucleases like DFF40 (DNA fragmentation factor 40; synonyms: CAD (Caspase-activated DNase), DFFB (DNA fragmentation factor subunit beta)), ENDOG (Endonuclease G), and DNASE1L3, while indirect mechanisms involve genetic modification, chemical inhibition, protein depletion, mutation, synthetic misregulation of (among others) upstream regulators of the above mentioned endonucleases like DFF45 (DNA fragmentation factor 45; synonyms: ICAD (Inhibitor of caspase-activated DNase), DFFA (DNA fragmentation factor subunit alpha)), Caspase 3, Caspase 7, granzyme B, and Caspase 9.

Accordingly, in specific embodiments, inhibition of DNA fragmentation encompasses direct or indirect inhibition of endonucleases by way of genetic modification.

Respective genetic modifications that can achieve inhibition of DNA fragmentation, e.g., by way of one or more of the above mechanisms, encompass a mechanism, selected from the group consisting of
(a) knockout of one or more genes, selected from the group consisting of CASP3 gene, DFF40 gene, and ENDOG gene,
(b) expression of one or more RNA silencing elements directed against one or more genes, selected from the group consisting of CASP3 gene, DFF40 gene, and ENDOG gene,
(c) expression of RNAseH activating elements or antisense RNA oligonucleotides blocking translational initiation or elongation of the group consisting of CASP3 gene, DFF40 gene, and ENDOG gene
(d) expression of one or more dominant negative mutants of caspase-3, DFF40, and/or ENDOG gene,
(e) strong overexpression of wildtype or caspase-resistant DFF45,
(f) expression of intrabodies and similar devices targeting Caspase-3, DFF40, and/or mitochondrial endonuclease G on the protein level,
(g) transcriptional silencing at the promoters of Caspase-3, DFF40, and/or ENDOG,
(h) epigenetic silencing by targeted DNA methylation or histone modifications of promoters from the group consisting of CASP3 gene, DFF40 gene, and ENDOG gene,
(i) exon skipping via U7 smOpt, antisense oligonucleotides (AON) or splice switching oligonucleotides (SSO) on DFF40, CASP3 or ENDOG pre-mRNA, and
(j) inhibitory isoform shifting, e.g. of DFF45 by reduction of ASF/SF2.

Respective means of gene knockout, the expression of RNA silencing elements, the expression of dominant negative mutants of specific proteins, the overexpression of e.g. wildtype or caspase-resistant DFF45, the expression of intrabodies, transcriptional silencing, epigenetic silencing by DNA methylation or histone modifications, exon skipping via U7 smOpt, antisense oligonucleotides (AON) or splice switching oligonucleotides (SSO), and inhibitory isoform shifting are not particularly limited and are known in the art. By way of example, the expression of RNA silencing elements can, e.g., include the expression of suitable microRNAs (miRNA), small interfering RNAs (siRNA), piwi-interacting RNA (piRNA) or repeat associated small interfering RNA (rasiRNA), as known in the art.

In other specific embodiments, inhibition of DNA fragmentation encompasses direct or indirect inhibition of endonucleases by way of chemical inhibition.

According to such embodiments, the inhibition of DNA fragmentation is not effected endogenously, i.e., by way of a genetic modification of the cells in which AAV is produced, but exogenously, i.e., by way of exposing the cells in which AAV is produced to an inhibitor of DNA fragmentation, e.g. (i) an inhibitor of apoptotic, parthanatotic and/or pyroptotic pathways, or (ii) an inhibitor of Caspase 3/DFF40-induced DNA strand breakage, during the AAV production phase.

Chemical inhibitors of DNA fragmentation are not particularly limited and are known in the art. However, in preferred embodiments of all of the above aspects of the present invention, the inhibitor of DNA fragmentation is selected from the group consisting of Z-VAD-fmk (Z-Val-Ala-Asp fluoromethyl ketone; CAS No. 161401-82-7); Z-IETD-fmk (Z-Ile-Glu-Thr-Asp; CAS. No. 210344-98-2); Z-DEVD-fmk (Z-Asp-Glu-Val-Asp; CAS Nr. 210344-95-9); PNR-3-80 (5-((1-(2-naphthoyl)-5-chloro-1H-indol-3-yl)methylene)-2-thioxodihydro-pyrimidine-4,6(1H,5H)-dione); PNR-3-82 (5-((1-(2-naphthoyl)-5-methoxy-1H-indol-3-yl)methylene)-2-thioxodihydropyrimidine-4,6(1H,5H)-dione); Zn²⁺ ions; EDTA (ethylenediaminetetraacetic acid); adezmapimod (SB 203580; 4-(4-fluorophenyl)-2-(4-methylsulfinylphenyl)-5-(4-pyridyl)-1H-imidazole; CAS No. 152121-47-6); and proteolysis-targeting chimeras (PROTACs) directed against caspase-3, DFF40, and/or mitochondrial endonuclease G, wherein Z-VAD-fmk is particularly preferred.

Respective chemical inhibitors can be added to the cells in an AAV production process from five days prior to the start of AAV production until two days after start of AAV production. Suitable concentration ranges are not particularly limited and are known in the art. This includes e.g. a range of 0.5 to 20 μg/ml, preferably of 10 to 20 0.5 to 20 μg/ml, for for Z-VAD-fmk, Z-IETD-fmk, or Z-DEVD-fmk.

In this context, PROTACs are heterobifunctional small molecules composed of two active domains and a linker, capable of removing specific unwanted proteins. PROTACs work by inducing selective intracellular proteolysis, and consist of two covalently linked protein-binding molecules, one capable of engaging an E3 ubiquitin ligase, and another that binds to a target protein meant for degradation. Recruitment of the E3 ligase to the target protein results in ubiquitination and subsequent degradation of the target protein via the proteasome.

In certain embodiments, DNA fragmentation is inhibited by way of overexpression of one or more enzymes involved in DNA repair, e.g. DNA repair enzymes, and/or enzymes that inhibit DFF40. Enzymes relevant in this respect include PARP1 (Poly [ADP-ribose] polymerase 1), DNA Ligase IV,BRCA1, BRCA2, FEN1, Ligase III, MRE11, NBS1, and XRCC1.

In a third aspect, the present invention relates to a method for producing AAV, comprising the step of producing AAV in a cell line as defined above for the first and second aspects of the present invention.

Methods for producing AAV in a given cell line are not particularly limited and are known in the art.

In a fourth aspect, the present invention relates to a method of producing Adeno-associated virus (AAV), comprising the step of exposing the cells in which AAV is produced to an inhibitor of DNA fragmentation during the AAV production phase.

According to this aspect of the present invention, the inhibition of DNA fragmentation is not effected endogenously, i.e., by way of a genetic modification of the cells in which AAV is produced, but exogenously, i.e., by way of exposing the cells in which AAV is produced to an inhibitor of DNA fragmentation, e.g. (i) an inhibitor of apoptotic, parthanatotic and/or pyroptotic pathways, or (ii) an inhibitor of Caspase 3/DFF40-induced DNA strand breakage, during the AAV production phase.

Chemical inhibitors of DNA fragmentation are not particularly limited and are known in the art. However, in preferred embodiments of all of the above aspects of the present invention, the inhibitor of DNA fragmentation is as defined above.

Respective chemical inhibitors can be added to the cells in an AAV production process from five days prior to the start of AAV production until two days after start of AAV production. Suitable concentration ranges are not particularly limited and are known in the art. This includes e.g. a range of 0.5 to 20 μg/ml, preferably of 10 to 20 μg/ml, for Z-VAD-fmk, Z-IETD-fmk, or Z-DEVD-fmk, as defined above.

Gene editing techniques known in the art provide full irreversible gene knockout or gene modifications, allowing the generation of new fully stable gene knockout and site specific genetically modified cell lines.

In the present invention, direct or indirect inhibition of endonucleases by way of chemical inhibition, as defined above for the first, second, and third aspect of the present invention, on the one hand, and direct or indirect inhibition of endonucleases by way of genetic modification, as defined above for all aspects of the present invention, on the other hand, are particularly preferred.

As used herein, the term “comprising”/“comprises” expressly includes the terms “consisting essentially of”/“consists essentially of” and “consisting of”/“consists of”, i.e., all of said terms are interchangeable with each other herein.

Further, as used herein, the term “about” preferably represents a modifier of the specified value of ±10%, more preferably ±8%, ±6%, ±5%, ±4%, ±3%, ±2%, ±1%, or ±0.5%. Thus, by way of example, the term “about 100” can include the ranges of 90 to 110, 92 to 108, 94 to 106, 95 to 105, 96 to 104, 97 to 103, 98 to 102, 99 to 101, or 99.5 to 100.5.

The present invention has surprisingly identified DNA fragmentation as the major source of nuclease-resistant product-related contamination by host cell DNA.

The present invention advantageously prevents or reduces the packaging of host cell DNA during the production of the AAV particles in the host cell and thus significantly reduces nuclease-resistant product-related contamination. Specifically, the present invention prevents or reduces nuclease-resistant contamination of AAV products while these are still being produced and without affecting the production of the AAV particles in the host cell, wherein by way of inhibiting DNA fragmentation, the packaging of host cell DNA is reduced.

During the production of AAV particles in human host cells, the production process itself kills the host cell. This death occurs, among other things, through programmed cell death.

The packaging of wild-type AAV genomes occurs in the nucleus of infected cells and is mediated by the AAV Rep78 protein that bridges the Rep binding element (RBE) located on the vector genome-associated Inverted Terminal Repeats (ITRs) to sites proximal to pores on the surface of preformed AAV empty capsids, thus mediating the translocation of single-stranded DNA vector genomes into the preformed capsids. However, Rep78 expression results in the activation of caspase-3 and induces apoptotic cell death and disruption of the cell cycle.

Apoptosis is a form of programmed cell death that occurs in multicellular organisms and is governed by a set of cysteinyl-aspartate specific proteases (caspases) that can be triggered by intrinsic and extrinsic stimuli. Once activated, caspases execute the apoptotic cell death in an irreversible cascade. The main morphological changes of the apoptotic process include cell and nuclear shrinkage, chromatin condensation, internucleosomal cleavage of chromosomal DNA, formation of apoptotic bodies and phagocytosis by neighboring cells.

During apoptosis, host cell DNA is degraded by so-called endonucleases into defined pieces. The process of DNA fragmentation is controlled, among other things, by the cleavage of ICAD (inhibitor of caspase activated DNase) activated by caspase-3 and the subsequent activation of nuclease CAD (caspase activated DNase; DFF40). The cleavage of genomic DNA by these endonucleases takes place specifically between the nucleosomes and produces DNA fragments with a length of about 180 base pairs and multiples thereof.

Apoptotic DNA fragmentation is mediated by DNA fragmentation factor (DFF) which is a heterodimeric protein composed of DFF45 (ICAD) and DFF40 (CAD). DFF40 contains an intrinsic DNase activity, while DFF45 serves as an inhibitor of DFF40 activity. Following activation of apoptosis, caspase-3 cleaves DFF45 and DFF45 dissociates from DFF40 which cleaves DNA into oligonucleosome sized fragments (180 bp intervals) known as “apoptotic ladder” when visualized by agarose gel electrophoresis.

Thus, the present invention advantageously reduces product-related impurities, affording increased product quality. Moreover, in doing so, the present invention improves viral genome packaging resulting in an increased percentage of full capsids. Furthermore, the present invention increases host cell viability, resulting in an increase of productivity.

Unlike process-related impurities, i.e., non-encapsidated DNA contaminations that can be strongly reduced by downstream purifications, encapsidated host cell DNA cannot be removed without destruction of the product itself. Encapsidated DNA is, however, the more hazardous type of impurity as it is efficiently shuttled into the target cell's nucleus. This can have multiple negative side effects:
1. Oncogenesis: Random integration of producer cell-derived DNA in a patient cell might disrupt tumor suppressor genes, perturb cell cycle control and cause uncontrolled cell growth. Conversely, transfer of functional DNA sequences from producer cells (e.g. promotor elements or DNA segments encoding miRNAs) might activate dormant DNA sequences or deregulate the transcriptome.
2. Immunotoxicity: Random integration into coding sequences can cause de novo expression of immune reactive neo-epitopes (with and without frame shifting).
3. Apoptotic cell death: free DNA activates DNA damage response, which can lead to apoptosis—a particular problem in neurodegenerative diseases where the very treatment is supposed to prevent apoptosis.
4. Inflammatory responses e.g. mediated by intracellular dsDNA sensing via cGAS-STING pathway.

The figures show:

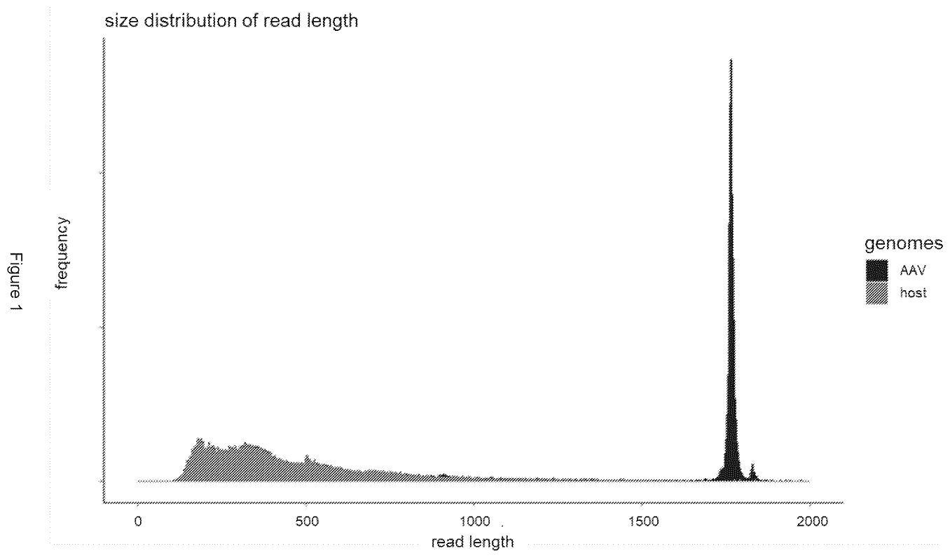

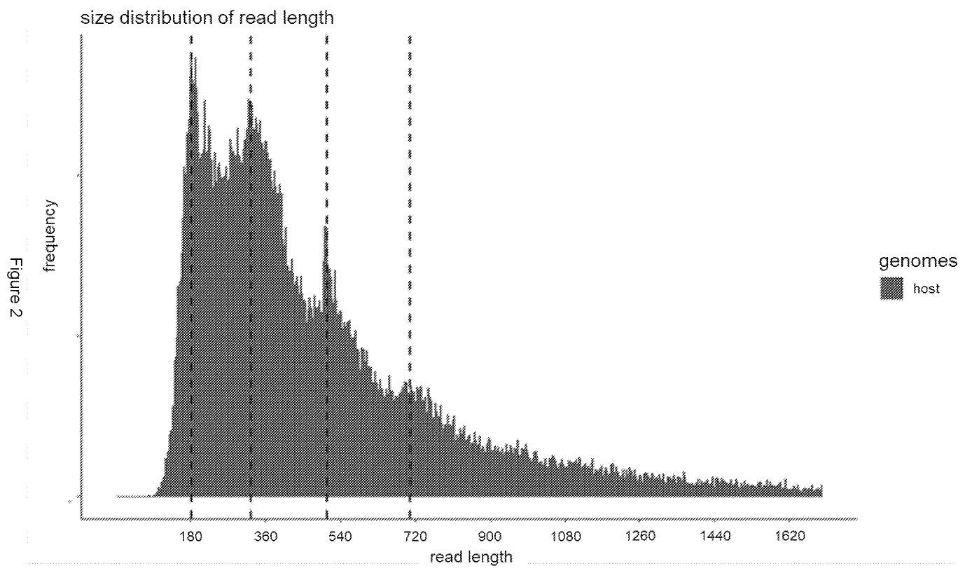

FIG. 1:

Fragment length distribution of AAV encapsidated DNA that align to a) host cell DNA or b) rAAV genome (1800 bp).

Y-axis represents the frequency of fragments, the x-axis the corresponding read length in bp.

FIG. 2:

Fragment length distribution of AAV encapsidated DNA that aligns to host cell DNA.

Y-axis represents the frequency of fragments, the x-axis the corresponding read length in bp. Dashed lines represent the maximum of individual peaks within the fragment distribution. (peak1=181 bp, peak2=325 bp, peak3=507 bp, peak4=708 bp).

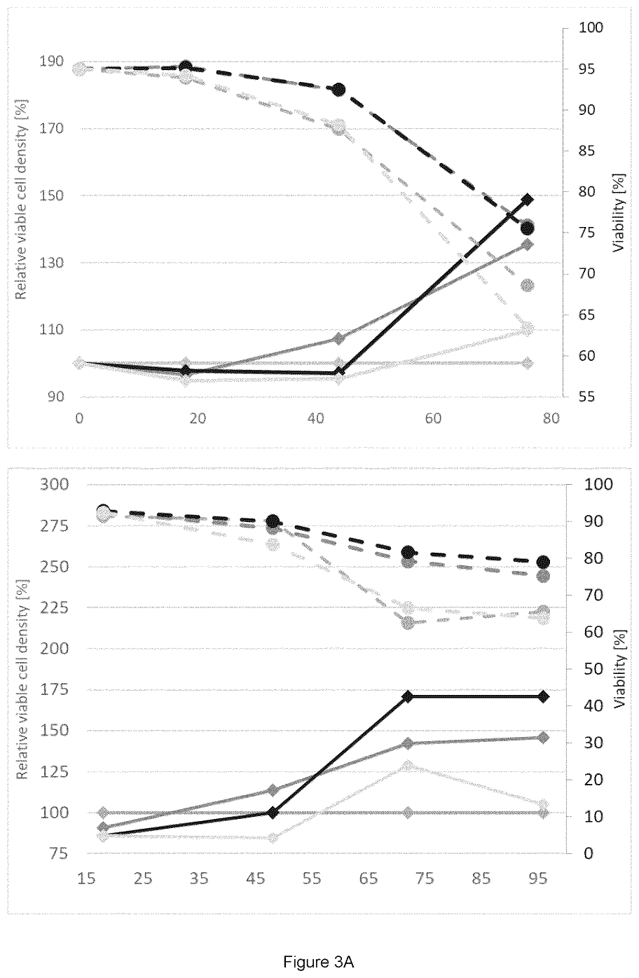

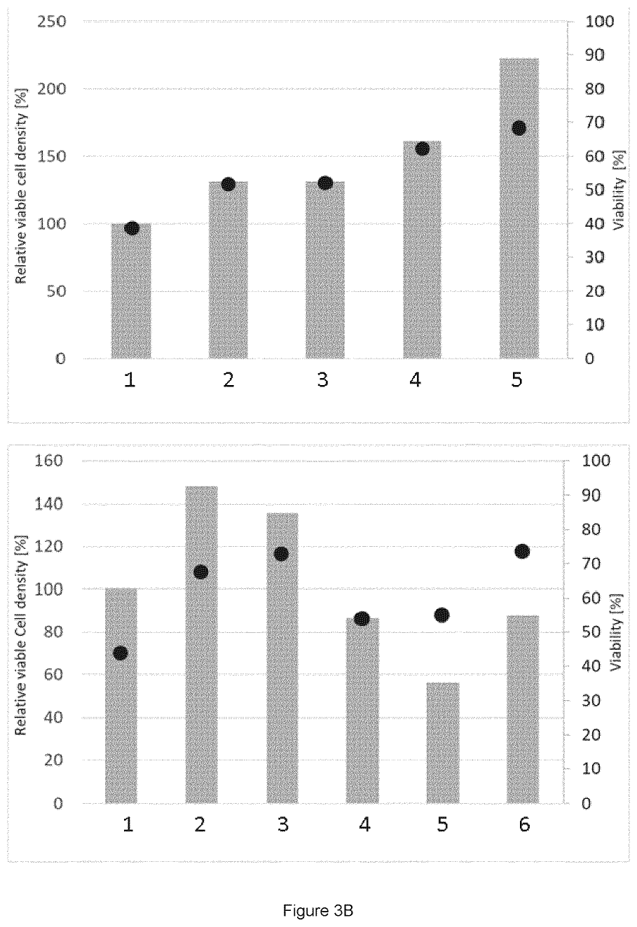

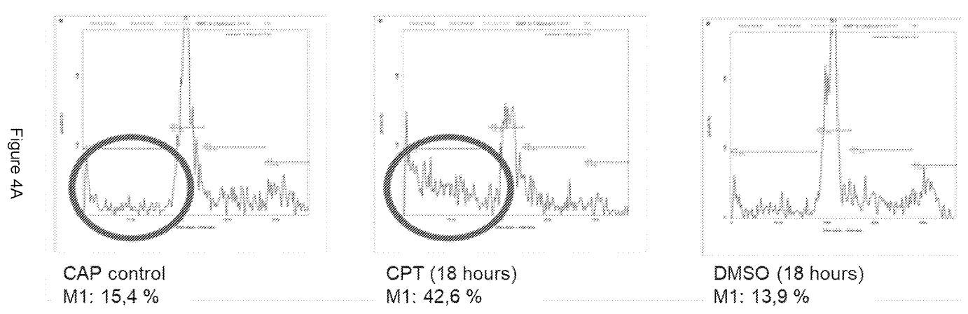

FIG. 3:

Viability of induced stable producer clone 1 and stable producer clone 2 at different concentrations of Z-VAD-fmk.
a) Stable producer clone 1 (upper panel) and 2 (lower panel). Relative viable cell density (solid diamonds) and viability (solid circles) measured at different time points post induction and supplementation [hours]. Z-VAD-fmk concentrations ranging from 10 μg/ml (black) over 2.5 μg/ml (dark grey) to 0 μg/ml (light grey) and DMSO treated (lightest grey).
b) Upper panel: Relative viable cell density (grey bars) and viability (black dots) at 4 days post induction/transfection of a CAP Alpha cell line in a semi-transient AAV producer setting using different concentrations of Z-VAD-fmk. 1: no Z-VAD-fmk; 2: 2 μM: 3: 5 μM; 4:10 μM; 5:20 μM Z-VAD-fmk. Lower panel: a stable packaging pool without transfection of a cargo plasmid. Measured at 72 h post transfections. Viabilities indicated by black circles.1: no Z-VAD-fmk; 2:10 μM Z-VAD-fmk; 3:20 μM Z-VAD-fmk; 4: DMSO; 5:10 μM Camptothecin; 6:10 μM Camptothecin+20 μM Z-VAD-fmk

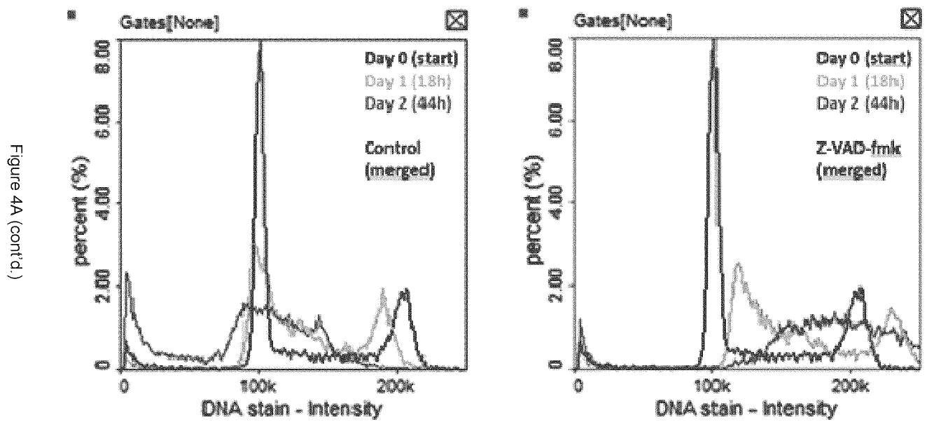

FIG. 4:

Inhibition of apoptotic DNA fragmentation reduces amount and packaging of hcDNA.
a) First page: sensitivity test for Sub-G1 DNA-fragmentation assay (Chemometec) using Camptothecin treatment (10 μM) on parental CAP cells over night. M1 population as % of global DNA stain. Second page: merged curves for apoptotic DNA fragmentation determined by same assay after 0 h (black curve), 18 h (dark grey), and 44 h (light grey), with Z-VAD-fmk (right) and in control samples (left, induction only)
b) First page: Relative share of encapsidated hcDNA [%] in transient and stable AAV8 production at 3 days post induction (transient transfection) and 5 days post induction (stable clone). Odd-numbered bars: no Z-VAD-fmk; even-numbered bars: 10 μg/ml Z-VAD-fmk; 1-6: CAP cells with transient transfection: 1+2: Rep/Cap+cargo 1; 3+4: Rep/Cap+cargo 2; 5+6: Rep/Cap, no cargo. 7+8: HEK293 transiently transfected with Rep/Cap+cargo 1; 9+10: stable producer clone 1, no transfection; 11: Background assay control. Encapsidated hcDNA was measured in relation to the respective untreated controls, each set at 100%. Second page: fold difference of hcDNA concentration over time [hours] in an untreated AAV production using stable producer clone 1 (light grey line) normalized to 10 μg/ml of Z-VAD-fmk (dark grey line) determined by ALU qPCR.
c) First page: Relative Capsid levels determined by ELISA in stable producer clone 1 and transiently transfected HEK293 suspension cells at different Z-VAD-fmk concentrations and compared with Camptothecin. Samples harvested at 5 days post induction. 1: untreated control; 2: HEK293+20 μM Z-VAD-fmk; 3: HEK293+40 μM Z-VAD-fmk; 4: stable producer clone 1+20 μM Z-VAD-fmk; 5: stable producer clone 1+40 μM Z-VAD-fmk; 6: stable producer clone 1+DMSO; 7: stable producer clone 1+10 μM Camptothecin. Second page: corresponding host cell DNA per capsid as determined by ALU qPCR normalized to untreated controls. Relative shares indicated above bars. 1: untreated control; 2+3: HEK293; 2:20 μM of Z-VAD-fmk; 3:40 μM Z-VAD-fmk; 4-7: stable producer clone 1; 4:20 μM Z-VAD-fmk; 5:40 μM Z-VAD-fmk; 6: DMSO; 7:10 μM Camptothecin.

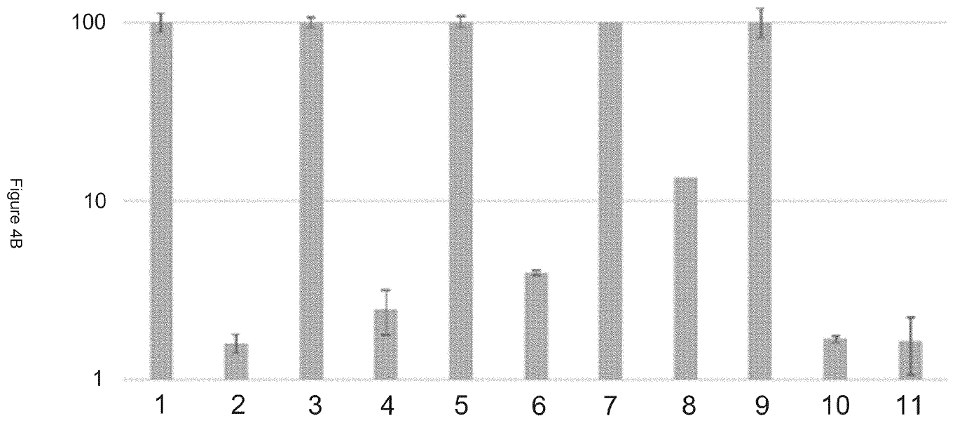

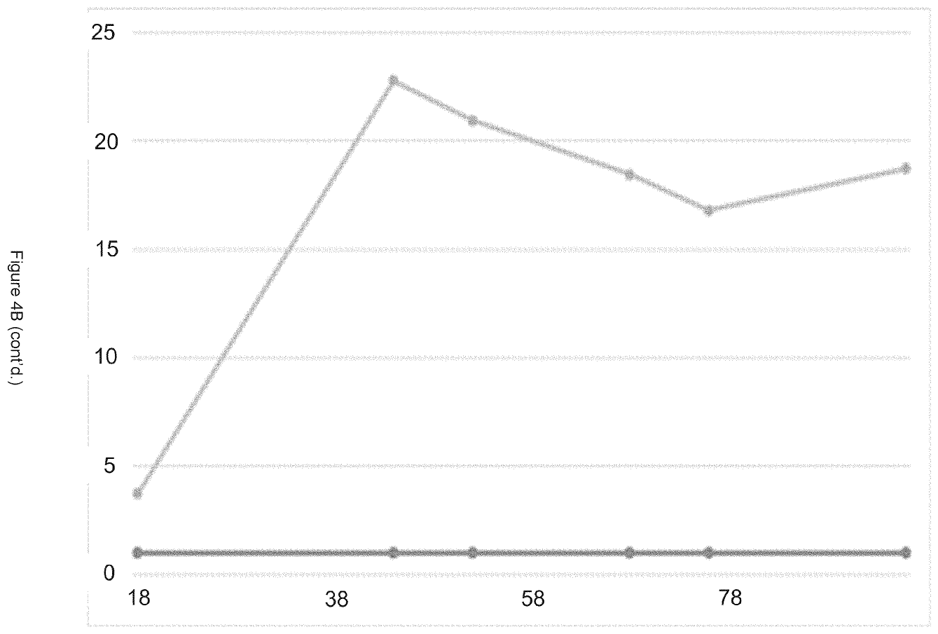

FIG. 5:

Ratios of viral genome containing capsids upon treatment with Z-VAD-fmk.

Influence of Z-VAD-fmk on semi-transient transfection of a stable packaging pool or full transient transfection of CAP cells. Vg/ml measured by qPCR and normalized to untreated samples (grey bars) and viral genomes normalized to amount of capsid determined by ELISA (black diamonds).
Odd-numbered: untreated controls; Even-numbered: 20 μM Z-VAD-fmk; 1-4: AAV8 packaging pool; 1+2: cargo 1; 3+4: cargo 2; 5-8: CAP (full transient); 5+6: cargo 1, 7+8: cargo 2

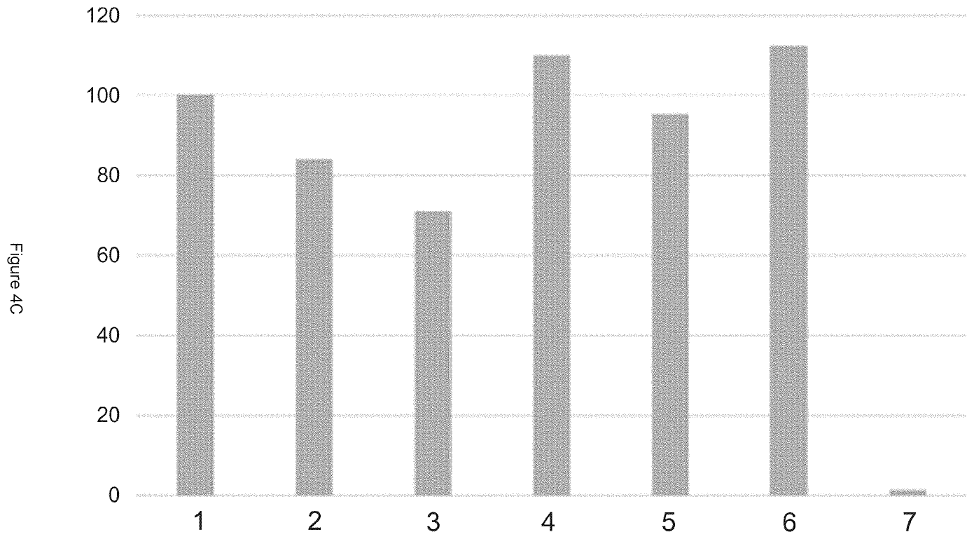

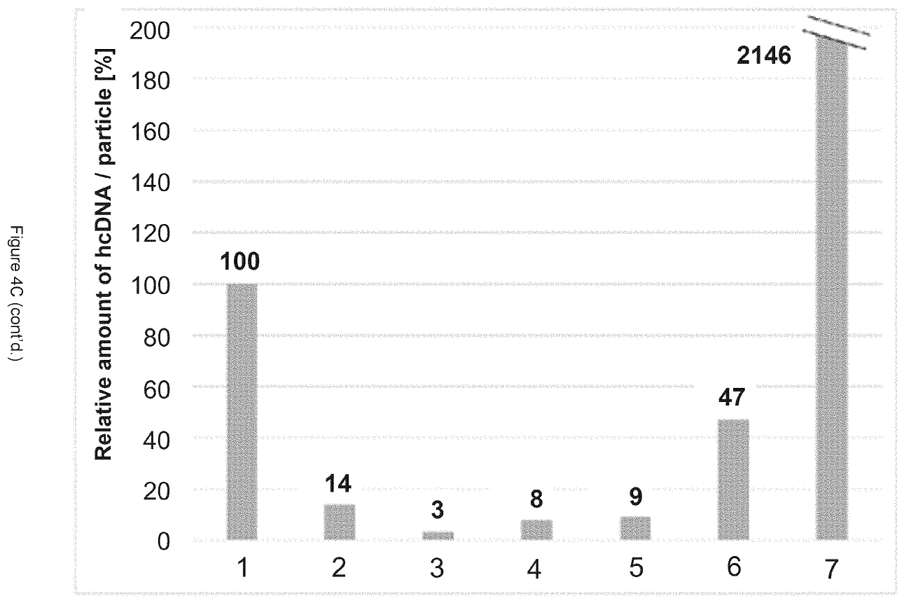

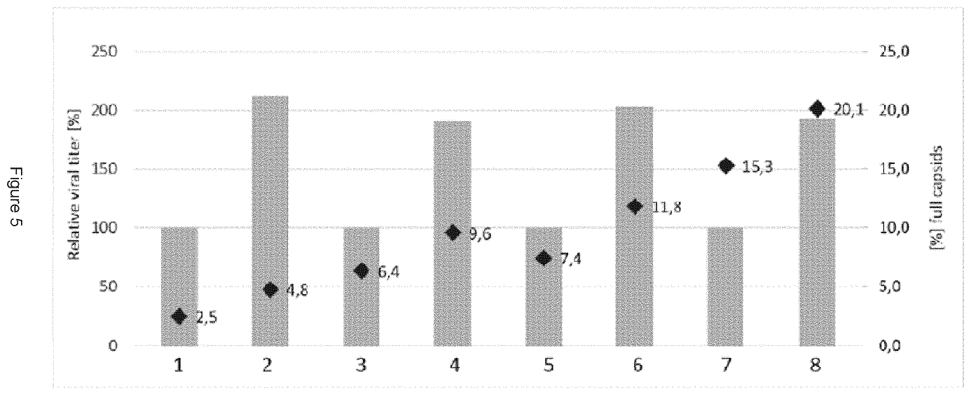

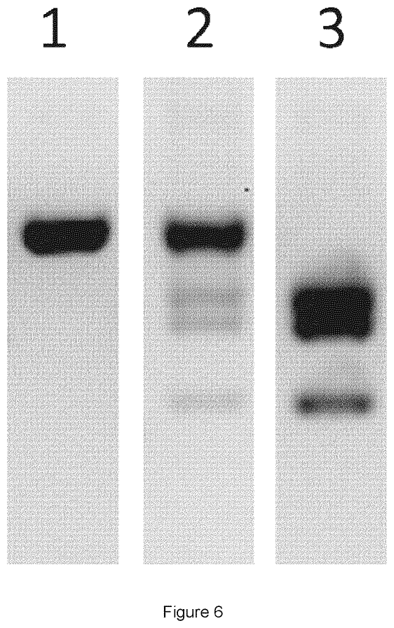

FIG. 6:

Knockout of DFF40 in AAV producer cell line.

PCR amplicon of DFF40 gene from DNA of AAV producer cell line expressing wildtype DFF40 (lane 1). PCR amplicon of same segment in pool of knockout cells based on same producer cell line (lane 2). DFF40 alleles with internally deleted sequences visible as several low-molecular weight bands. Clonal cell line derived from knockout pool without detectable amplicon corresponding to DFF40 wildtype allele (lane 3).

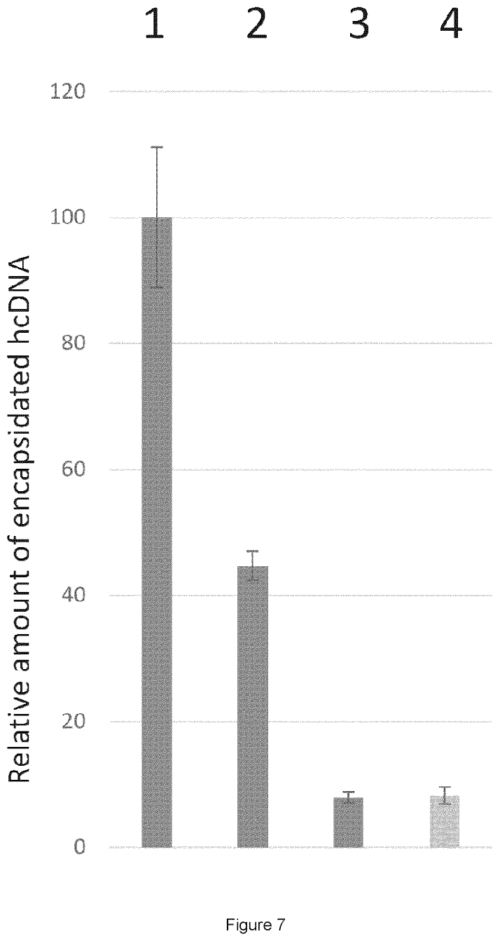

FIG. 7:

Quantification of encapsidated hcDNA

Relative amount of encapsidated hcDNA in DFF40-WT AAV producer cell line (lane 1), DFF40 knockout pool (lane 2) and DFF40 knockout single cell clone (lane 3). Amount of hcDNA indistinguishable from background control (lane 4).

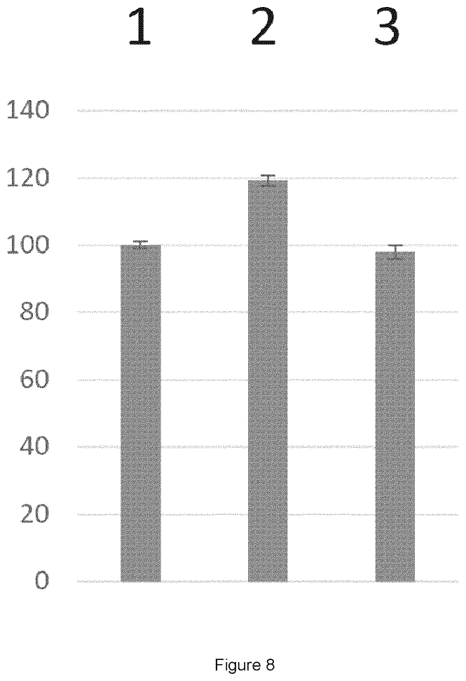

FIG. 8:

Viral titers of DFF40 KO cells remain unchanged

Normalized titers of packaged viral genomes [%] compared between DFF40-WT AAV producer cell line (lane 1), DFF40 knockout pool (lane 2) and DFF40 knockout single cell clone (lane 3).

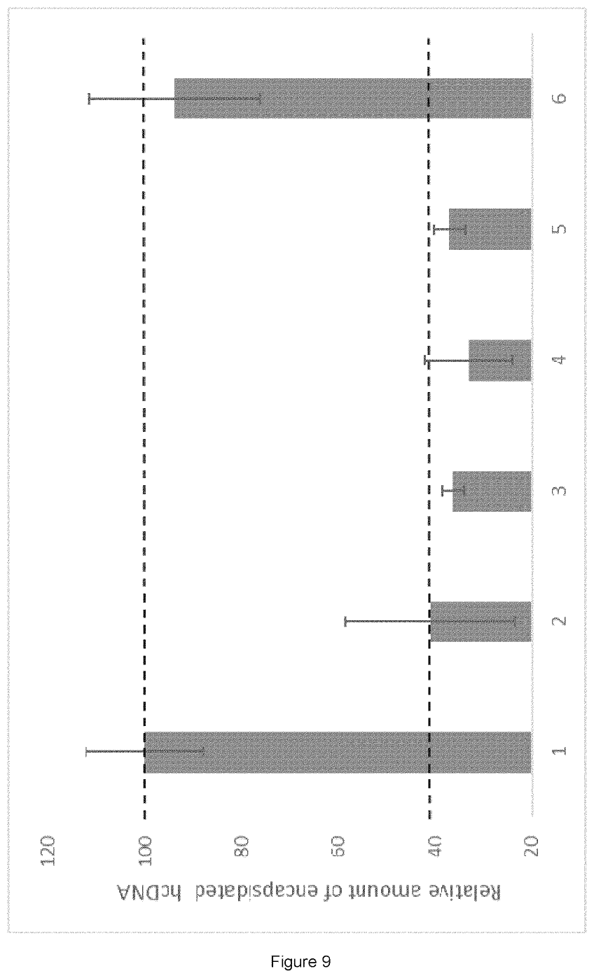

FIG. 9:

Knockdown of DFF40 and other apoptotic genes by siRNA

Relative amount of encapsidated hcDNA from stable AAV producer cells transfected with siRNAs targeting the genes DFF40 (3), CASP3 (4), CASP8 (5) and AIFM1 (6) normalized to mock transfected control (1) and compared to untransfected but ZVAD-fmk treated cells (2).

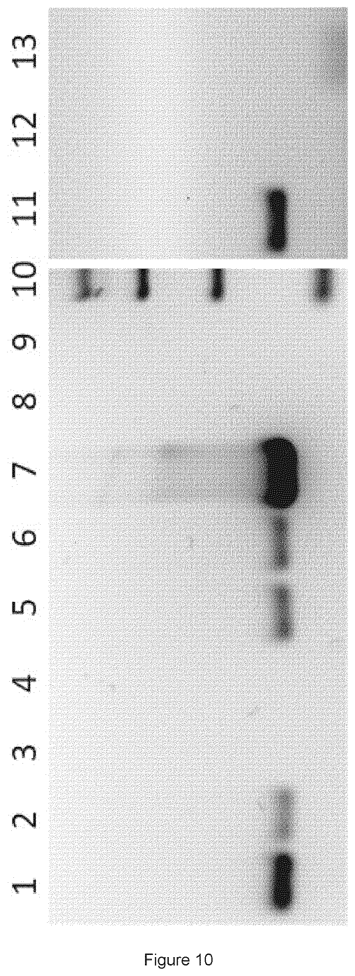

FIG. 10:

Endpoint PCR for detection of encapsidated hcDNA

HEK293 wt cells (1-4), DFF40 KO pools (5-6) and DFF40 KO single cell clones (11, 12, 13) were transfected with either cargo 3 (1, 3, 5) or cargo 1 (2, 4, 6, 11, 12, 13) and either treated with ZVAD-fmk (3, 4) or left untreated (1, 2, 5, 6, 11, 12, 13). Purified HEK293 DNA was used as PCR control (7, 8) either with DNAse treatment (8) or without (7, 11), whereas non-template control (9) was used as PCR negative control. DNA marker 1 kb plus (ThermoFisher) (10).

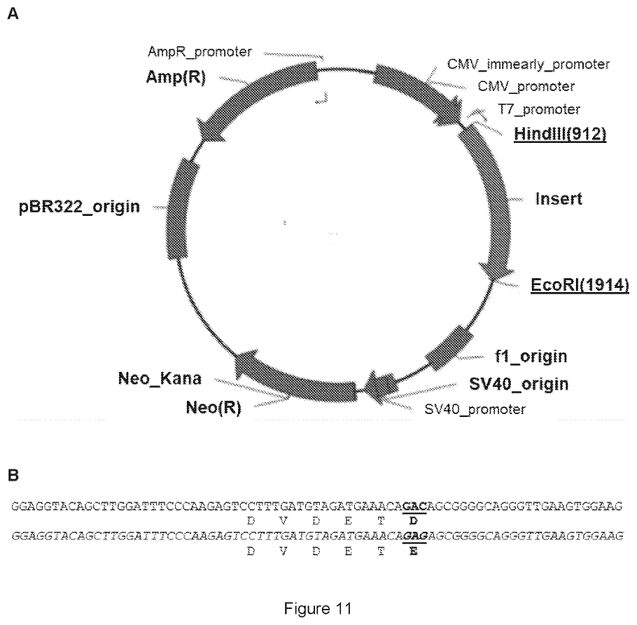

FIG. 11:

Overexpression plasmid constructs

Schematic representation of the overexpressing constructs used. (A) Wild-type PARP1 (NM_001618.4) and a single mutant form of DFF45 (NM_004401.3) (D117E) were cloned into the mammalian expression vector pcDNA3.1(+) (ThermoFischer), which is marked as “insert” in the schematic representation. (B) Mutation in the DFF45 gene on amino acid position 117 from aspartic (D; GAC (SEQ ID NOs: 9 and 10)) acid to glutamic acid (E; GAG (SEQ ID NOs: 11 and 12)) leads to a caspase-resistant isoform.

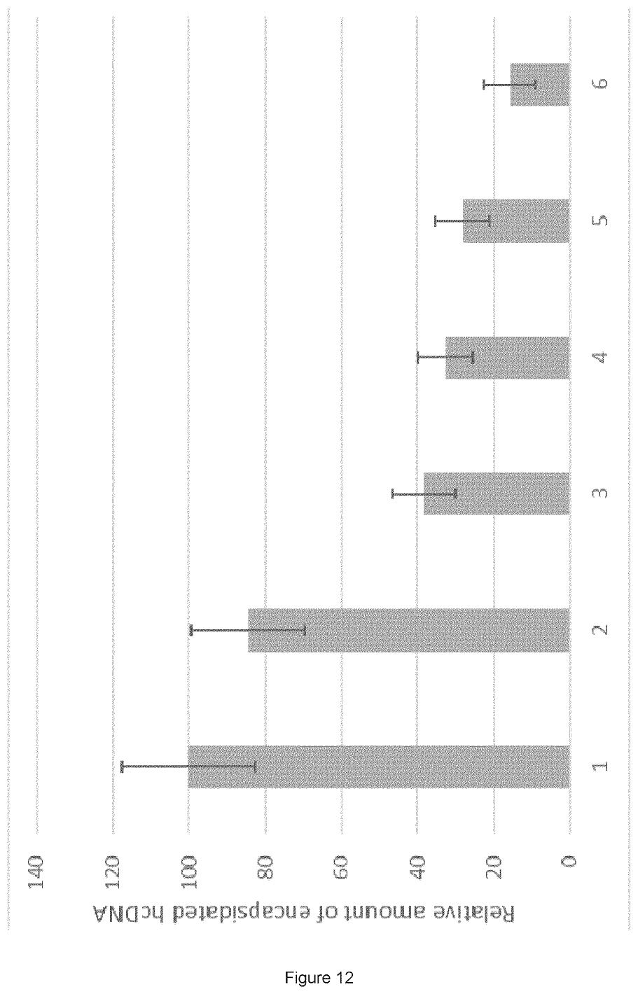

FIG. 12:

Overexpression of antiapoptotic genes to reduce hcDNA encapsidation in AAV production

Stable inducible AAV producer cells were transfected with an empty expression plasmid (2), a plasmid encoding PARP1 (3) or DFF45 (D117E) (4). Encapsidated hcDNA was measured and compared to non-transfected cells that were either uninduced (6) or induced (1,5) and Z-VAD-fmk treated (5).

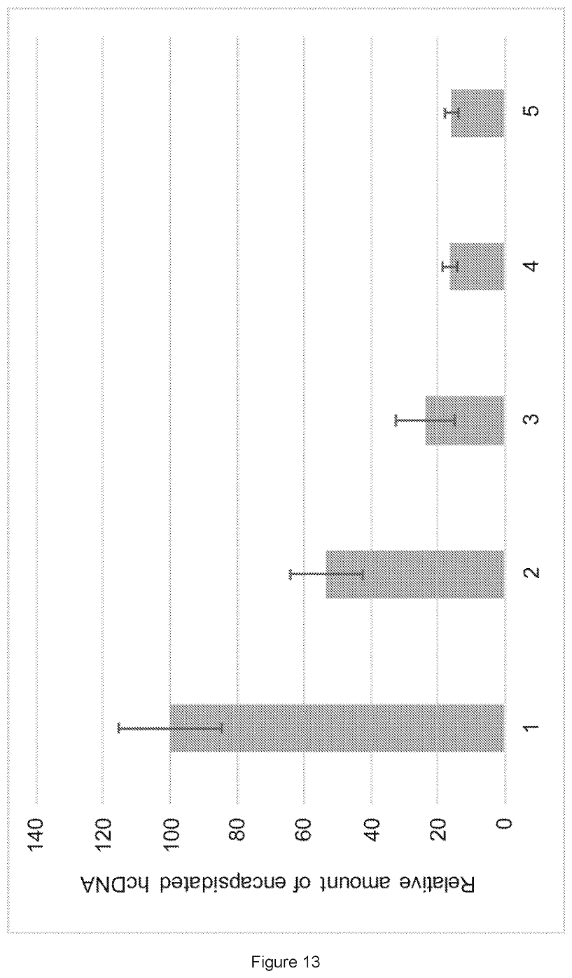

FIG. 13:

Inhibition of hcDNA encapsidation using MAPK-inhibitor

Compared with vehicle treated controls (DMSO, 1), treatment of stable inducible AAV producer cells with p38 inhibitor Adezmapimod reduced hcDNA encapsidation when applied at 30 μM (2) and 60 μM (3) final concentration. The effect at 60 μM was comparable to Z-VAD-fmk-treatment (4) and uninduced samples (5).

The present invention relates to the following nucleotide sequences:

SEQ ID NO: 1
SV40 PolyA Primer forward
5′-AGCAATAGCATCACAAATTTCACAA-3′
SEQ ID NO: 2
SV40 PolyA Primer reverse
5′-CCAGACATGATAAGATACATTGATGAGTT-3′
SEQ ID NO: 3
SV40 PolyA Probe FAM-ZEN-IBFQ
5′-AGCATTTTTTTCACTGCATTCTAGTTGTGGTTTGTC-3′
SEQ ID NO: 4
hcDNA Alu Primer forward
5′-GAGGCGGGCGGATCA-3′
SEQ ID NO: 5
hcDNA Alu Primer reverse
5′-CCCGGCTAATTTTTGTATTTTTAGTAG-3′
SEQ ID NO: 6
hcDNA Alu Probe HEX-ZEN-IBFQ
5′-CAGCCTGGCCAACATGGTGAAACC-3′
SEQ ID NO: 7
DFF40 Primer forward
5′-TTAGAACGGCCCGCAGTC-3′
SEQ ID NO: 8
DFF40 Primer reverse
5′-CCTTGAGACCCGAGAGTTCAC-3′
SEQ ID NO: 9
Partial DFF45 wildtype coding sequence
5′-GGAGGTACAGCTTGGATTTCCCAAGAGTCCTTTGATGTAGATGAAA
CAGACAGCGGGGCAGGGTTGAAGTGGAAG-3′
SEQ ID NO: 10
Partial DFF45 wildtype amino acid sequence
DVDETD
SEQ ID NO: 11
Partial DFF45 mutated coding sequence
5′-GGAGGTACAGCTTGGATTTCCCAAGAGTCCTTTGATGTAGATGAAA
CAGAGAGCGGGGCAGGTTGAAGTGGAG-3′
SEQ ID NO: 12
Partial DFF45 mutated amino acid sequence
DVDETE
SEQ ID NO: 13
CASP3 Primer forward
5′-AATGGACCTGTTGACCTGAAA-3′
SEQ ID NO: 14
CASP3 Primer reverse
5′-CACGGCAGGCCTGAATAA-3′
SEQ ID NO: 15
AIFM1 Primer forward
5′-GACTGGTGGCCTGGAAATAG-3′
SEQ ID NO: 16
AIFM1 Primer reverse
5′-CGCCTCCTTCCCAACTTTAT-3′
SEQ ID NO: 17
CASP8 Primer forward
5′-GTCTGTGCCCAAATCAACAAG-3′
SEQ ID NO: 18
CASP8 Primer reverse
5′-GAGTCCGAGATTGTCATTACCC-3′
SEQ ID NO: 19
DFF40 Primer forward
5′-CCTGGAACCTGGATCACATAAT-3′
SEQ ID NO: 20
DFF40 Primer reverse
5′-TACTCCCAGTCCACTTCTCT-3′
SEQ ID NO: 21
GAPDH Primer forward
5′-AAGGTGAAGGTCGGAGTCAA-3′
SEQ ID NO: 22
GAPDH Primer reverse
5′-AATGAAGGGGTCATTGATGG-3′
SEQ ID NO: 23
Endpoint PCR Primer forward
5′-GACTGTGGTTGCTTCATGCT-3′
SEQ ID NO: 24
Endpoint PCR Primer reverse
5′-ACGTGAATGGTCTTCAGCAG-3′

The present invention will be further illustrated by the following example without being limited thereto.

### EXAMPLES

### Experimental Procedures

### Long-Read Sequencing Using PacBio:

rAAV samples were treated with Denarase and purified by affinity chromatography.

PacBio Long-read sequencing was performed as known in the art. Briefly, encapsidated DNA was purified, DNA ends were repaired, and barcode adapters ligated. Damaged regions were repaired, the libraries were purified, and primers were annealed. Finally, the DNA polymerase was bound, and the DNA fragments were sequenced on the PacBio Sequel platform with v2.0 chemistry.

### Bioinformatic Analysis Workflow:

PacBio raw data were demultiplexed using PacBio SMRT Link
Circular consensus sequencing (CCS) reads were generated by PacBio SMRT Link
Alignment of CCS reads to reference using pbmm2
Parsing of BAM files using samtools and custom scripts
Counting and visualization of CCS reads was performed using R/Bioconductor

### Cell Culture:

Four Different Types of Producer Cells were Used:
1. Parental CAP or HEK293 cells, which carry adenoviral E1A and E1B genes and which were transiently transfected with genes encoding the AAV Cap proteins VP1, VP2, and VP3; genes encoding the AAV Rep proteins Rep78, Rep68, Rep52, and Rep40; genes encoding the adenoviral helper functions E4orf6, E2A and VA-RNA and the gene of interest (GOI) flanked by AAV inverted terminal repeat sequences (ITRs) for AAV production (fully transient approach).
2. “Alpha”-cells are derivatives of parental cells and harbor additional stably inserted and inducible genes encoding the AAV Rep proteins Rep78, Rep68, Rep52, and Rep40 and genes encoding the adenoviral helper functions E4orf6, E2A and VA-RNA. Upon transfection of a capsid-encoding plasmid alongside a cargo sequence, Alpha cells are enabled to produce AAVs (semi-transient system).
3. “Packaging” cells originate from Alpha cells and additionally carry stably integrated inducible genes for the AAV Cap proteins VP1, VP2, and VP3.
4. “Full producers”, are derived from packaging cells and additionally carry the gene of interest (GOI) flanked by AAV inverted terminal repeat sequences (ITRs) thus having stably integrated all genes necessary to produce AAVs.

CAP cells were routinely cultivated in chemically defined, serum-free PEM medium (Thermo Fisher Scientific) supplemented with 4 mM GlutaMax (Gibco) in shake flasks (125 mL; Corning) on a shaking incubator at 120 rpm (5 cm orbit), 5% CO₂ and 37° C.

During routine cultivation, cells were diluted with fresh medium to a viable cell density of 0.5-1×10⁶ cells/ml every 72 to 96 h. Viable cell density and viability were determined by trypan blue exclusion using a Vi-Cell Blu cell counter (Beckman Coulter).

### Transient Transfection

Transient transfection was performed using PEImax (PolySciences) and 1 pg plasmid per cell in FreeStyle F17 medium (Thermo Fisher Scientific) with 4 mM Glutamax and 50 μl/L IGF. AAV production was induced by adding doxycycline (Clontech) to a final concentration of 1 μg/mL. AAV8 serotype expression was under control of a doxycycline inducible promoter and cargo plasmids contained AAV2-ITRs flanking a GFP reporter construct. Plasmids were transfected at a 1:1 ratio. Stable producer cells were induced by addition of doxycycline without further transfection. Caspase inhibitor Z-VAD-fmk (InvivoGen) and P38 MAPK inhibitor Adezmapimod (SB 203580, CellSignal) were added using the indicated concentrations at time point of induction.

At different time points post transfection, cell suspension was harvested, and cells were lysed by addition of Triton-X 100 (Sigma). After centrifugation, supernatants were diluted and incubated with TurboDNase (ThermoFisher Scientific), in order to remove non-encapsidated DNA contaminants. After inactivation of TurboDNAse, proteinase K was added to digest the viral capsid and subsequently amplify viral genomic DNA or hcDNA via ddPCR or qPCR.

qPCR/ddPCR to Determine Viral Titer and Host Cell DNA:

The following primer/dual-labelled probe combinations (ordered at MWG, Eurofins and IDT, respectively, Table 1) directed against
a) the SV40 PolyA (quantification of viral titer) or
b) Alu sequences (quantification of hcDNA)
were used:

As standard for the viral genome, linearized transgene plasmid with a defined copy number was used. As standard for the hcDNA assay previously isolated CAP and HEK293 DNA (purified with DNeasy® Blood & Tissue Kit) was used.

TABLE 1
Primer/Probe combination for qPCR and ddPCR assays

| Primer/Probe | Sequence |
|---|---|
| SV40 PolyA Primer for | 5′-AGCAATAGCATCACAAATTTCACAA-3′ |
| (SEQ ID NO: 1) | |
| SV40 PolyA Primer rev | 5′-CCAGACATGATAAGATACATTGATGAGTT-3′ |
| (SEQ ID NO: 2) | |
| SV40 PolyA Probe | 5′-AGCATTTTTTTCACTGCATTCTAGTTGTGGTTTGTC-3′ |
| FAM-ZEN-IBFQ | |
| (SEQ ID NO: 3) | |
| hcDNA_Alu_fwd | 5′-GAGGCGGGCGGATCA-3′ |
| (SEQ ID NO: 4) | |
| hcDNA_Alu_rev | 5′-CCCGGCTAATTTTTGTATTTTTAGTAG-3′ |
| (SEQ ID NO: 5) | |
| hcDNA_Alu_Probe | 5′-CAGCCTGGCCAACATGGTGAAACC-3′ |
| HEX-ZEN-IBFQ | |
| (SEQ ID NO: 6) | |

AAV samples were treated with Turbo DNAse (Thermo Fischer Scientific) to digest the non-encapsidated DNA before the capsids were digested using Proteinase K. Proteinase K was inactivated by a heating step at 95° C. for 15 min. The samples were measured either by qPCR or ddPCR as follows:

The qPCR reaction contained the following components: 2×Brilliant Multiplex qPCR Master Mix (Agilent), nuclease-free H₂O (Thermo Fisher Scientific), primer/probe mix and sample/standard. qPCR was run on an LightCycler 480 according to the manufacturer's instructions.

In addition, ddPCR was used for absolute quantification of viral titer. Prior to droplet formation, samples were diluted in sample dilution buffer (GeneAmp 10×PCR Buffer I (Thermo Fischer Scientific), Sheared Salmon Sperm (SSS) DNA, Pluronic F-68 non-ionic surfactant) and added to the 2×ddPCR Supermix (BioRad) with the appropriate primers/probes (cf. Table 1). Droplets were generated using the Biorad QX200 Droplet Generator according to the manufacturer's instructions. PCR was performed in the Biorad C100 Touch™ thermal cycler. Droplets were counted using the BioRad QX200 Droplet Reader and analyzed using QuantaSoft Analysis Software.

### DNA Fragmentation Assay

A DNA fragmentation assay by Chemometec was applied using the NucleoCounter® NC-3000™ according to note No. 3003. Rev. 1.4.

In brief, cells were permeabilized with Ethanol, which allows low molecular weight DNA fragments (e.g., apoptotic DNA) to leak out, while large fragments and undamaged chromosomes are retained inside the nucleus. Total DNA content was stained by DAPI and detected using the NucleoCounter® NC-3000™. Here, cells typically fall into one of three clusters when plotted by signal intensity. The G1 peak represents the majority of cells with 2n DNA content. A second, smaller peak represents cells in G2 phase of the cell cycle, containing 4n. An intermediate spread-out population between the G1 and G2 peak represents cells in S-phase while cells with apparent sub-G1 DNA content are indicative of apoptotic DNA fragmentation and can be discerned due to ethanol-induced release of apoptotic fragments.

### Cell Line and Cultivation

A CAP-cell derived stable AAV producer cell line was used for the experiments in Examples 5 to 7. The cell line expresses doxycycline inducible adenoviral Helper genes, AAV replicase, capsid sequences and an ITR-flanked transgene.

CAP cells were routinely cultivated in chemically defined, serum-free PEM medium (Thermo Fisher Scientific) supplemented with 4 mM GlutaMax (Gibco) in shake flasks (125 mL; Corning) on a shaking incubator at 120 rpm (5 cm orbit), 5% CO₂ and 37° C.

During routine cultivation, cells were diluted with fresh medium to a viable cell density of 0.5-1×10⁶ cells/ml every 72 to 96 h. Viable cell density and viability were determined by trypan blue exclusion using a Vi-Cell Blu cell counter (Beckman Coulter).

### AAV Production

AAV production was induced by adding doxycycline (Clontech) to a final concentration of 1 μg/mL. Cell suspension was harvested after three days, and cells were lysed by addition of Triton-X 100 (Sigma). After centrifugation, supernatants were diluted and incubated with TurboDNase (ThermoFisher Scientific), in order to remove non-encapsidated DNA contaminants. After inactivation of TurboDNAse, proteinase K was added to digest the viral capsid and subsequently amplify viral genomic DNA or hcDNA via ddPCR or qPCR.

### Gene Knockouts

Gene Knockouts for DFF40 were conducted as known in the art.

### Single Cell Cloning

The mixed polyclonal pool of knockout and wildtype cells was single cell cloned by the single cell dispenser “c.sight” from Cytena and monitored for monoclonality by the Cell Metric Imager “Solentim”.

### Knockdown

Transfections of siRNAs directed against DFF40 (s17440), CAPS3 (s397), CAPS8 (s2427), or AIFM1 (s17440); (all ThermoFischer) were conducted using FuGene-SI transfection reagent (Biozol) according to the manufacturer's instructions. One day after transfection, cells were induced with doxycycline and harvested three days later.

Knockdown Efficiency was Determined by qPCR:

RNA from CAP cells (10⁶) was isolated using a Quick-RNA Microprep kit (Zymo Research) according to the manufacturer's instructions. Residual genomic DNA was digested with DNase I (Thermo Fisher Scientific) for 15 min at 37° C. After enzyme inactivation for 10 min at 65° C. in the presence of 2.5 mM EDTA, 50 ng RNA per reaction were subjected to cDNA synthesis and quantitative PCR in a LightCycler 480 device (Roche) using a Quant-X One-Step qRT-PCR TB Green Kit (Takara Bio) with gene-specific primers (Integrated DNA Technologies) according to the manufacturer's instructions.

TABLE 2
Primers used for RT-PCR

| Primer | Sequence |
|---|---|
| CASP3 FWD | 5′-AATGGACCTGTTGACCTGAAA-3′ |
| (SEQ ID NO: 13) | |
| CASP3 REV | 5′-CACGGCAGGCCTGAATAA-3′ |
| (SEQ ID NO: 14) | |
| AIFM1 FWD | 5′-GACTGGTGGCCTGGAAATAG-3′ |
| (SEQ ID NO: 15) | |
| AIFM1 REV | 5′-CGCCTCCTTCCCAACTTTAT-3′ |
| (SEQ ID NO: 16) | |
| CASP8 FWD | 5′-GTCTGTGCCCAAATCAACAAG-3′ |
| (SEQ ID NO: 17) | |
| CASP8 REV | 5′-GAGTCCGAGATTGTCATTACCC-3′ |
| (SEQ ID NO: 18) | |
| DFF40 FWD | 5′-CCTGGAACCTGGATCACATAAT-3′ |
| (SEQ ID NO: 19) | |
| DFF40 REV | 5′-TACTCCCAGTCCACTTCTCT-3′ |
| (SEQ ID NO: 20) | |
| GAPDH_fwd | 5′-AAGGTGAAGGTCGGAGTCAA-3′ |
| (SEQ ID NO: 21) | |
| GAPDH_rev | 5′-AATGAAGGGGTCATTGATGG-3′ |
| (SEQ ID NO: 22) | |

### Overexpression

Wild-type PARP1 (NM_001618.4) and a single mutant form of DFFA (NM_004401.3) (D117E) were cloned into the mammalian expression vector pcDNA3.1(+) (ThermoFischer) and transfected into stable inducible AAV producer cells. One day after transfection, AAV production was induced and cells were harvested on day 3 post induction.

Endpoint PCR for Detection of hcDNA Encapsidation

Encapsidated hcDNA was prepared as described. PCR setup involved 3 μl sample as template for an endpoint PCR with 40 cylces using Q5 High-Fidelity DNA polymerase (NEB) according to the manufacturer's instructions. PCR samples were analyzed on a 2% agarose gel.

### Primers Endpoint PCR:

Endpoint-fw
(SEQ ID NO: 23)
5′-GACTGTGGTTGCTTCATGCT-3′
Endpoint-rv
(SEQ ID NO: 24)
5′-ACGTGAATGGTCTTCAGCAG-3′

### Example 1

### Analysis of Nuclease-Resistant DNA in Recombinant AAV Particles

### Introduction

The fragment length of DNA encapsidated into recombinant AAV particles mapping to a) host cell genome or b) to rAAV genome was determined. For this, purified and DNaseI treated rAAV particles were analyzed using the PacBio technology.

### Results:

PacBio analysis revealed a distinct separation of DNA aligned to host cell genome and the rAAV genome in terms of fragment length. The rAAV genome showed an average fragment length of approximately 1800 bp that matched with the actual size of the ITR flanked region. The size of DNA that maps to host cell genome showed a broader range starting at approximately 100 bp (FIG. 1).

The detailed analysis of the host cell DNA fragment length in FIG. 2 revealed a wave-like pattern with distinct peaks. The maxima of each peak (visualized by dashed lines) matched to the size distribution of the theoretical size distribution of the apoptotic ladder with an averaged deviation of +/−20 bp as shown in Table 3.

TABLE 3
Comparison of the theoretical DNA fragment length after
apoptotic fragmentation and of the actual host cell
DNA fragment length (calculated by peak maximas) present
in the viral particle analyzed by PacBio.

| DNase protected | Apoptotic | Peak maxima in PacBio | Deviation |
|---|---|---|---|
| nucleosomes | ladder [bp] | seq [bp] | [bp] |
| 1 | 180 | 181 | +1 |
| 2 | 360 | 325 | −35 |
| 3 | 540 | 507 | −33 |
| 4 | 720 | 708 | −12 |

These data lead to the assumption that the source of encapsidated host cell DNA in this AAV production is the fragmentation of DNA by the apoptotic pathway.

### Example 2

Inhibition of apoptosis by pan-caspase inhibitor Z-VAD-fmk increases the relative number of viable cells after induction.

### Introduction

Various stable and transient AAV producer cells were induced with doxycycline and treated with different concentrations of the antiapoptotic pan-caspase inhibitor Z-VAD-fmk.
Cell viability was evaluated using the NucleoCounter NC-3000.

### Results

Treatment of cells with Z-VAD-fmk increased viable cell density and viability. The observed effects correlated with applied concentrations and increased viability by ˜10 percentage points as compared to untreated cells over the course of 80 h of treatment with 10 μg/ml of Z-VAD-fmk (FIG. 3a) and independently of the investigated CAP cell line. In contrast, pro-apoptotic agent Camptothecin reduced viability and VCD (FIG. 3b), an effect that could be compensated by co-culturing with anti-apoptotic Z-VAD-fmk.

### Example 3

Pan-caspase inhibitor Z-VAD-fmk drastically reduces amount of hcDNA in AAV particles.

### Introduction

Z-VAD-fmk inhibits caspases 3 and 8 which are upstream of the apoptotic nucleases DFF40 and ENDOG, respectively.
Z-VAD-fmk treatment should therefore reduce chromosomal fragmentation and decrease amount of packageably sized AAV cargo.
Transiently transfected CAP cells and HEK293 cells or full producers were cultured to produce AAV in the presence of absence of Z-VAD-fmk.
Resulting AAV particles were harvested from cell suspension on days 1, 2, 3, and 4 post induction.
Encapsidated host cell DNA was measured using a dedicated qPCR assay amplifying ALU repeats.
DNA fragmentation was determined using the DNA fragmentation assay by Chemometec.

### Results

Z-VAD-fmk reduced the amount of low molecular weight DNA that otherwise appears during vector production. Conversely, the analogous increase in short DNA fragments upon treatment with proapoptotic Camptothecin supports apoptotic origin of this type of DNA (FIG. 4A).

Analyses of viral particles revealed that treating cells with up to 40 μg/ml Z-VAD-fmk dramatically decreased packaging of host cell DNA compared to untreated controls (FIGS. 4B and 4C). These results are consistently observed with the fully transient approach and full producers and are obtained with CAP as well as HEK293 cells.

ELISA readouts confirmed that reduced hcDNA levels are not the consequence of an overall decreased capsid production under Z-VAD-fmk treatment. On the contrary, in stable producer cells capsid levels were even slightly increased in the presence of Z-VAD-fmk and normalization to capsid levels (ELISA) revealed a ˜10-fold reduction in encapsidated hcDNA compared to controls (FIG. 4C).

### Example 4

Pan-caspase inhibitor Z-VAD-fmk increases ratio of viral genome containing capsids to empty capsids.

### Introduction

Most recombinant AAV capsids remain devoid of viral genomes after assembly.
Separation of full and empty particles is a major challenge in downstream processing of viral particles.
Improving encapsidation of viral genomes would greatly reduce manufacturing costs and increase the quality of the final product.

### Results

An AAV8 packaging pool and parental CAP cells were transfected with cargo plasmids and AAV production plasmids, respectively. Quantification of viral titers was performed using qPCR, capsid levels were determined using ELISA. Treatment with Z-VAD-fmk increased the proportion of viral genome-containing capsids by 30 to 90% in transient and semi-transient transfection approaches with two different cargo plasmids (FIG. 5).

### Example 5

Knockout of DFF40 on the DNA level.

### Introduction

In order to prevent apoptotic fragmentation of the host cell's DNA, DFF40 was knocked out as known in the art. PCR amplification across the targeted region revealed knockout efficiency.

### Results

Endpoint PCR of wildtype control samples (lane 1), polyclonal pools (lane 2) and a DFF40 clonal cell line (lane 3) display different proportions of knockout. While a single band corresponding to the expected genomic amplicon size appears on agarose gel for the wildtype sample, a clear reduction in the intensity of this band, as well as the emergence of lower molecular weight bands was detected in the polyclonal knockout pool (FIG. 6). After single cell cloning, several cell lines could be isolated that had no remaining DFF40 wildtype alleles left.

### Example 6

Reduction of encapsidated hcDNA by DFF40 knockout.

### Introduction

DFF40 is a key factor in apoptotic DNA fragmentation. Knockout of DFF40 should therefore prevent fragmentation of DNA into low molecular weight fragments and hence keep viral capsids free of hcDNA contamination derived from the respective AAV producer cell.

### Results

Comparative quantification via qPCR revealed a relative reduction of about 60% in the DFF40 polyclonal knockout pool (FIG. 7, lane 2) compared to the hcDNA levels detected in AAV preparations from the corresponding producer cell line harboring the DFF40 wildtype alleles (FIG. 7, lane 1). Genetically confirmed clonal knockout cell lines (FIG. 7, lane 3) derived from this KO pool showed a reduction of hcDNA contamination down to background levels (FIG. 7, lane 4).

### Example 7

### Vector Production in DFF40 Knockout Cells

### Introduction

DFF40 is expressed in AAV producer cells and evolutionarily conserved, therefore a knockout might have detrimental impact on AAV production.

### Results

Cell viability of DFF40 knockout clones and corresponding polyclonal pools was unchanged and vector titers did not decline in response to the genetic alteration (FIG. 8, lanes 1-3).

### Example 8

Knockdown of DFF40 and Other Apoptotic Genes by Transfection of siRNA

### Introduction

Knockout of DFF40 copies on the DNA level leads to a complete loss of DFF40 protein. However, such full depletion of DFF40 might not be required in order to reduce encapsidation of hcDNA. Short interfering RNA (siRNA) is known to reduce protein levels by targeting the corresponding mRNA and thereby inducing a partial depletion of these transcripts. However, less than one in four randomly selected siRNAs silence their cognate target by more than 95%.
Stable AAV producer cells were induced with doxycycline and treated with siRNA targeting various genes that are part of the apoptotic DNA fragmentation pathway.
DFF40: endonuclease involved in the cell apoptotic process that facilitates the DNA breakup. DFF40 cleaves dsDNA at internucleosomal linker regions in chromatin into high molecular weight fragments (20-100 kb) and then into oligonucleosomal fragments known as low molecular weight (LMW) DNA degradation and visualized as a laddering pattern in an agarose gel electrophoresis (DNA ladder).
CASP3: CASP3 directly targets ICAD for degradation.
CASP8: initiator CASP8 mediates CASP3 activation.
AIFM1: AIFM1 induces high molecular weight DNA fragmentation by triggering chromatin condensation, but not the subsequent low molecular weight DNA fragmentation.
One day after transfection, cells were induced by addition of doxycycline and 3 days post induction cells were harvested and analyzed for encapsidation of hcDNA.
Encapsidated host cell DNA was measured using a dedicated qPCR assay amplifying ALU repeats.

### Results

Stable inducible AAV producer cells were transfected with siRNAs directed against the mRNA of DFF40 (knockdown efficiency (KD-Eff) 66%), CASP3 (KD-Eff 61%, CASP8 (KD-Eff 78%) and AIFM1 (KD-Eff 92%). Viral particles derived from cells transfected with siRNA targeting DFF40, CASP3, CASP8 had a similar >60% reduction of hcDNA as compared to mock transfected cells while transfections with siRNA against AIFM1, which is known to not fragment DNA on low molecular level, had no effect on hcDNA content (FIG. 9).

Therefore, effective inhibition of hcDNA packaging can be achieved not only by full depletion of DFF40 using gene engineering but likewise by a more moderate reduction of DFF40 levels using RNA silencing. Furthermore, the positive effect is not limited to DFF40 as such but can also be achieved by knockdown of upstream factors in the apoptotic cascade, which are also known to be involved in DNA degradation to low molecular weight fragments. Fragmentation of DNA into high molecular weight fragments achieved by AFM1 depletion is not sufficient to reduce hcDNA packaging.

### Example 9

### Knockout of DFF40 in HEK293 Cells

### Introduction

Knockout of DFF40 as well as treatment with Z-VAD-fmk prevented hcDNA encapsidation during AAV production in human CAP cells.
Treatment of HEK cells with Z-VAD-fmk leads to similar reduction of encapsidated hcDNA as CAP in a transient setting (cf. Example 3).
Therefore, DFF40 was also knocked out in HEK293 cells in order to demonstrate the effect of a complete loss of DFF40 in an independent cellular background.
Encapsidated host cell DNA was measured using an endpoint PCR assay amplifying a genomic DNA sequence from HEK293 cells.

### Results

HEK293 knockouts were generated using the same approach as for CAP cells. HEK293 wt cells (1-4), DFF40 KO pools (5-6) and DFF40 KO single cell clones (11, 12) were transfected with either cargo3 (1, 3, 5) or cargo 1 (2, 4, 6, 11, 12) and either treated with ZVAD-fmk (3, 4) or left untreated (1, 2, 5, 6, 11, 12). Highly sensitive PCR amplification of a genomic DNA sequence from HEK293 cells demonstrates encapsidation of adenoviral E1 sequences present in HEK293 wildtype cells transfected with cargo 1 or cargo 3, which was undetectable in samples from transiently transfected cells that were either treated with Z-VAD-fmk or represent confirmed single cell KO clones derived from a polyclonal pool of DFF40 KO cells. Encapsidated hcDNA detectable in the polyclonal pool (5, 6) was expected as KO efficiency is below 100% and end point PCR was designed to amplify even residual amounts of contaminating DNA (FIG. 10).

Knockout of DFF40 reduces or entirely prevents hcDNA encapsidation in CAP cells as well as HEK293 cells and therefore most likely any mammalian cell capable of AAV production.

### Example 10

Overexpression of Antiapoptotic Genes Prevents Encapsidation of hcDNA

### Introduction

Inhibition of apoptosis can be accomplished by interference with pro-apoptotic factors using knockdown, knockout and chemical inhibition as demonstrated above.
Overexpression of antiapoptotic genes on the other hand has been described to likewise inhibit or dampen programmed cell death.
A caspase-resistant version of DFF45 harboring the missense mutation D117E was shown to prevent DFF40-induced LMW fragmentation during apoptosis when transiently expressed in SH-SY5Y cells.
PARP1, an enzyme involved in DNA repair, is one of several known cellular substrates of caspases. Cleavage of PARP1 by caspase 3 is considered to be a hallmark of apoptosis. PARP1 was reported to be an antagonist of DNA fragmentation in the context of apoptosis as well as cell differentiation and gene expression.
Ectopic expression of caspase-resistant DFF45 mutant and PARP1 might reduce encapsidation of hcDNA in AAV production by reducing DNA fragmentation.
Stable AAV producer cell line was transfected with plasmids encoding either a dominant negative copy of DFF45 (D117E) or wt PARP1.
One day after transfection, cells were induced by addition of doxycycline and 3 days post induction cells were harvested and analyzed for encapsidation of hcDNA.
Encapsidated host cell DNA was measured using a dedicated qPCR assay amplifying ALU repeats.

### Results

Transfection of a stable AAV producer cell line with plasmids (FIG. 11) encoding either a dominant negative copy of DFF45 (D117E) (FIG. 12, lane 4) or wt PARP1 (FIG. 12, lane 3) successfully reduced hcDNA encapsidation by more than 60% compared to an untransfected control (lane 1). Despite an average transfection efficiency of only ˜40-60% in PEI approaches, ectopic expression of DFF45 (D117E) and PARP1 leads to similar effects on hcDNA encapsidation than the previously described treatment with Z-VAD-fmk (lane 5).

The results indicate that aside from inhibition, reduction and perturbation of pro-apoptotic factors, encapsidation of hcDNA can also be prevented by overexpression of antiapoptotic alleles as well as genes involved in DNA damage response during apoptosis, differentiation and gene expression.

### Example 11

### Chemical Inhibition of Upstream Pro-Apoptotic Factors by Non-Peptide Compounds

### Introduction

Pan-caspase inhibitor Z-VAD-fmk has been shown to successfully prevent apoptosis by inactivating all caspases and thereby inhibiting encapsidation of hcDNA. Other compounds like Z-IETD-fmk and Z-DEVD-fmk had a similar effect on encapsidation, yet compounds that are not based upon short peptides and targeting of caspases might achieve the same result.
Stable AAV producer cells were induced with doxycycline and treated with increasing concentrations of the p38 MAPK-inhibitor SB203580 (Adezmapimod). p38 mitogen-activated protein kinase (MAPK) has been described to induce apoptosis via activation of Bax and promoting cytochrome c release and caspase activation. Accordingly, p38 inhibitor Adezmapimod (SB203580) was shown to prevent stress-induced apoptosis and apoptotic induced DNA fragmentation.
Cell suspension was harvested at day 3 post induction and encapsidated hcDNA content was determined.
Encapsidated host cell DNA was measured using a dedicated qPCR assay amplifying ALU repeats.

### Results

The p38 MAPK inhibitor SB 203580 (FIG. 13, lanes 2 and 3) reduces the encapsidated hcDNA compared to the vehicle treated controls (lane 1). The reduction efficiency was at 60 μM of SB203580 (lane 3) was comparable to Z-VAD-fmk-treatment (lane 4) and uninduced samples (lane 5) and reached a reduction of more than 75% (FIG. 13).

The results indicate that effective prevention of hcDNA encapsidation by small drugs is not limited to inhibition of caspases or factors directly upstream of DFF40 but also encompasses proteins at the distal end of the apoptotic pathway.

1. A cell line in which DNA fragmentation is inhibited.

2. Use of a cell line in which DNA fragmentation is inhibited for the production of Adeno-associated virus (AAV).

3. The cell line according to claim 1, wherein the cell line is an AAV producer cell line.

4. The cell line according to claim 1, wherein the DNA fragmentation is (i) DNA fragmentation caused by programmed cell death, or (ii) DNA fragmentation caused by DNA strand breaks that are induced by Caspase 3/DFF40.

5. The cell line according to claim 1, wherein the DNA fragmentation is low molecular weight DNA fragmentation.

6. The cell line according to claim 5, wherein low molecular weight DNA fragmentation is DNA fragmentation resulting in DNA fragments of at most 20 kB length.

7. The cell line according to claim 1, wherein the genes encoding the components necessary for the production of AAV are transiently expressed in said cell line or are stably integrated into the cell genome.

8. The cell line according to claim 7, wherein the genes encoding the components necessary for the production of AAV are selected from the group consisting of genes encoding the AAV Cap proteins VP1, VP2, and VP3; genes encoding the AAV Rep proteins Rep78, Rep68, Rep52, and Rep40; genes encoding the adenoviral helper functions E4orf6, E2A, VA-RNA, and DBP; and genes encoding the Ad5 helper genes E1A and E1B; and the gene of interest (GOI) flanked by AAV inverted terminal repeat sequences (ITRs).

9. The cell line according to claim 7, wherein the following genes are transiently expressed in said cell line or are stably integrated into the cell genome:
the genes encoding the AAV Cap proteins VP1, VP2, and VP3;
a gene or genes encoding either one or both of the AAV Rep proteins Rep78 or Rep68; a gene or genes encoding either one or both of the AAV Rep proteins Rep52 or Rep40; the gene of interest (GOI) flanked by AAV ITRs; and
optionally, a gene or genes encoding either one, either two, either three, either four, or all of the adenoviral helper functions E1A, E1B, E4orf6, VA-RNA, DBP, and E2A.

10. The cell line according to claim 1, wherein the cell line is a cell line, selected from the group consisting of CAP cells, AGE1.hn, HEK293, PER.C6, NSO1, COS-7, BHK, CHO, CV1, VERO, HeLa, MDCK, BRL3A, W138, and HepG2 cells.

11. The cell line according to claim 1, wherein the DNA fragmentation is caused by programmed cell death, and wherein DNA fragmentation caused by programmed cell death is apoptotic DNA fragmentation, parthanatotic DNA fragmentation, and/or pyroptotic DNA fragmentation.

12. The cell line according to claim 1, wherein the DNA fragmentation is inhibited by way of direct or indirect inhibition of one or more endonucleases.

13. The cell line according to claim 12, wherein direct inhibition of one or more endonucleases encompasses
(i) genetic modification,
(ii) chemical inhibition, and/or
(iii) protein depletion,
of one or more endonucleases, selected from the group consisting of DFF40 (DNA fragmentation factor 40), ENDOG (Endonuclease G), and DNASE1L3.

14. The cell line according to claim 12, wherein indirect inhibition of one or more endonucleases encompasses
(i) genetic modification,
(ii) chemical inhibition,
(iii) protein depletion,
(iv) mutation, and/or
(v) synthetic misregulation,
of one or more upstream regulators of one or more endonucleases, wherein said upstream regulators are selected from the group consisting of DFF45 (DNA fragmentation factor 45), caspase-3, caspase-7, granzyme B, and caspase-9.

15. The cell line according to claim 1, wherein the DNA fragmentation is inhibited by way of a mechanism, selected from the group consisting of
(a) knockout of one or more genes, selected from the group consisting of CASP3 gene, DFF40 gene, and ENDOG gene,
(b) expression of one or more RNA silencing elements directed against one or more genes, selected from the group consisting of CASP3 gene, DFF40 gene, and ENDOG gene,
(c) expression of RNAseH activating elements or antisense RNA oligonucleotides blocking translational initiation or elongation of the group consisting of CASP3 gene, DFF40 gene, and ENDOG gene,
(d) expression of one or more dominant negative mutants of caspase-3, DFF40, and/or ENDOG gene,
(e) strong overexpression of wildtype or caspase-resistant DFF45,
(f) expression of intrabodies and similar devices targeting Caspase-3, DFF40, and/or mitochondrial endonuclease G on the protein level,
(g) transcriptional silencing at the promoters of Caspase-3, DFF40, and/or ENDOG,
(h) epigenetic silencing by targeted DNA methylation or histone modifications of promoters from the group consisting of CASP3 gene, DFF40 gene, and ENDOG gene,
(i) exon skipping via U7 smOpt, antisense oligonucleotides (AON) or splice switching oligonucleotides (SSO) on DFF40, CASP3 or ENDOG pre-mRNA, and
(j) inhibitory isoform shifting, e.g. of DFF45, by reduction of ASF/SF2.

16. The cell line according to claim 1, wherein DNA fragmentation is inhibited by way of overexpression of one or more enzymes, selected from the group consisting of enzymes involved in DNA repair, and enzymes that inhibit DFF40.

17. The cell line according to claim 16, wherein the enzymes are selected from the group consisting of PARP1 (Poly [ADP-ribose] polymerase 1), DNA Ligase IV, BRCA1, BRCA2, FEN1, Ligase III, MRE11, NBS1, and XRCC1.

18. A method for producing Adeno-associated virus (AAV), comprising the step of producing AAV in a cell line as defined in claim 1.

19. A method of producing Adeno-associated virus (AAV), comprising the step of exposing the cells in which AAV is produced to an inhibitor of DNA fragmentation during the AAV production phase.

20. The method according to claim 19, wherein the inhibitor of DNA fragmentation is selected from the group consisting of Z-VAD-fmk (Z-Val-Ala-Asp fluoromethyl ketone; CAS No. 161401-82-7); Z-IETD-fmk (Z-Ile-Glu-Thr-Asp; CAS. No. 210344-98-2); Z-DEVD-fmk (Z-Asp-Glu-Val-Asp; CAS No. 210344-95-9); PNR-3-80 (5-((1-(2-naphthoyl)-5-chloro-1H-indol-3-yl)methylene)-2-thioxodihydro-pyrimidine-4,6(1H,5H)-dione); PNR-3-82 (5-((1-(2-naphthoyl)-5-methoxy-1H-indol-3-yl)methylene)-2-thioxodihydropyrimidine-4,6(1H,5H)-dione); Zn²⁺ ions; EDTA (ethylenediaminetetraacetic acid); adezmapimod (SB 203580; 4-(4-fluorophenyl)-2-(4-methylsulfinylphenyl)-5-(4-pyridyl)-1H-imidazole; CAS No. 152121-47-6); and proteolysis-targeting chimeras (PROTACs) directed against caspase-3, DFF40 (DNA fragmentation factor 40), and/or mitochondrial endonuclease G.
